(19) United States
(12) Patent Application Publication
(10) Pub. No.: US 2026/0174557 A1
(43) Pub. Date: Jun. 25, 2026

(54) GUIDING DEVICE FOR FORMING INTERVENTIONAL CHANNEL, TRANSCATHETER INTERVENTIONAL SYSTEM AND PRE-EXPANSION METHOD

(71) Applicant: HEALCARDIO MEDTECH (HANGZHOU) CO., LTD., Hangzhou (CN)

(72) Inventor: Trung PHAM, Santa Rosa, CA (US)

(21) Appl. No.: 19/539,034

(22) Filed: Feb. 13, 2026

Related U.S. Application Data
(63) Continuation of application No. PCT/IB2024/057734, filed on Aug. 9, 2024.
(60) Provisional application No. 63/532,683, filed on Aug. 15, 2023.

(51) Int. Cl. A61F 2/24; A61B 17/34

(52) U.S. Cl. CPC A61F 2/2436; A61B 17/3439; A61F 2/2418; A61F 2/2427; A61F 2/2433

(57) ABSTRACT

A guiding device for forming an interventional channel includes an outer sheath being a radially deformable tube, a hemostatic valve connected to a proximal end of the outer sheath, and a driving member including a head and a transmission part. The driving member has an initial position and a working direction directed from the initial position toward the proximal end. The driving member moves along the working direction to expand the outer sheath until being withdrawn outside the human body.

### CROSS-REFERENCE TO RELATED APPLICATIONS

The present disclosure is a Continuation application of PCT Application No. PCT/IB2024/057734, filed on Aug. 9, 2024, which claims priority to U.S. Patent Application No. 63/532,683, filed on Aug. 15, 2023, the entire contents of which are hereby incorporated by reference.

### TECHNICAL FIELD

The technical field of the present disclosure relates to medical devices, in particular, to a guiding device for forming an interventional channel, a transcatheter interventional system and a pre-expansion method.

### BACKGROUND

In a transcatheter interventional procedure, the outer sheath is usually delivered through a puncture point first, to form a temporary channel in the human body, and then other interventional device among others are delivered through the temporary channel in sequence.

Taking the introduction of the outer sheath and the passage of subsequent interventional devices into account, the outer sheath needs to be pre-expanded in vivo.

In an existing pre-expansion operation, a pre-expansion member with a head is usually pushed from the proximal end to the distal end in the temporary channel, to radially expand the outer sheath through the head the outer diameter of which is slightly larger than the inner diameter of the outer sheath, and then the pre-expansion member is withdrawn, following with the other operations of the procedure.

The existing pre-expansion member and the pre-expansion method have problems of high push resistance and poor operating feel and the like.

### SUMMARY

The present disclosure provides a guiding device for forming an interventional channel, to reduce the operational difficulty of expanding the outer sheath.

The present disclosure provides a guiding device for forming an interventional channel, having opposing proximal and distal ends and including:
an outer sheath being a radially deformable tube;
a hemostatic valve connected to a proximal end of the outer sheath; and
a driving member including a head and a transmission part, the driving member having an initial position and a working direction directed from the initial position toward the proximal end. In the initial position, the head is outside the outer sheath and on a distal side of the outer sheath, and the transmission part is connected with the head and extends inside the outer sheath until being outside the proximal end of the outer sheath through the hemostatic valve. The head has a fixed shape, and when the driving member moves along the working direction, the head enters the outer sheath and drives a corresponding portion of the outer sheath to radially expand.

In the following, several alternatives are provided, but merely as further additions or preferences, instead of as additional limitations to the above-mentioned technical solution. Without technical or logical contradiction, the alternatives can be combined with the above-mentioned technical solution, individually or in combination.

In some embodiments, a tube wall of the outer sheath has a coiled structure, and has an expanded state in which the coiled structure is unfolded at a corresponding portion and a pre-expanded state in which the coiled structure is restored.

In some embodiments, the outer sheath is made of elastic material.

In some embodiments, a wall of the outer sheath includes reinforced fibers.

In some embodiments, the outer sheath includes an inner film layer, a middle layer and an outer film layer arranged sequentially from inside to outside in a radial direction.

In some embodiments, the thickness of the inner and outer film layers ranges from 0.01 mm to 0.5 mm, from 0.02 mm to 0.4 mm, or from 0.03 mm to 0.25 mm.

In some embodiments, the middle layer is made of reinforced fibers, and made by braiding a plurality of filaments.

In some embodiments, the outer sheath has relative to-be-expanded state and pre-expanded state, and the outer sheath in the pre-expanded state has a larger cross-sectional area and/or reduced radial tightening force than in the to-be-expanded state.

In some embodiments, when the driving member is in the initial position, the outer sheath is in the to-be-expanded state; and when the driving member moves along the working direction, a portion of the outer sheath corresponding to the head and a portion of the outer sheath on a distal side of the head are in the pre-expanded state.

In some embodiments, the outer sheath has a cross section in shape of a regular circle or ellipse, the head has a radial dimension D1, the outer sheath has an inner diameter D0 in the to-be-expanded state, and D1 is greater than D0.

In some embodiments, the hemostatic valve has a first channel for the head to pass through.

In some embodiments, the head includes a first crown at its proximal end, and the first crown continuously expands from the proximal end toward the distal end.

In some embodiments, in the initial position, a proximal end face of the first crown abuts against a distal opening of the outer sheath.

In some embodiments, the first crown has a chamfered or tapered outer peripheral surface.

In some embodiments, the head further includes a second crown at its distal end which tapers from a largest diameter of the first crown toward the distal end.

In some embodiments, the head further includes a second crown at its distal end, and a main body between the first crown and the second crown which extends in an equal diameter.

In some embodiments, the driving member further includes a guide portion located at a distal end of the head, and the guide portion tapers to its own distal end.

In some embodiments, the guiding device further includes a guide portion located at a distal end of the head, and the guide portion tapers to its own distal end;

The second crown has a greater inclination than the guide portion.

In some embodiments, the head is generally spherical or ellipsoidal.

In some embodiments, the head is provided with a lubricating layer on an outer periphery thereof.

In some embodiments, in the initial position, the lubricating layer extends to a contact portion of the head with the outer sheath.

In some embodiments, the driving member is formed by separate pieces and selected from one of the following arrangements:
a) the head and the transmission part are separate pieces in an axial direction;
b) the head includes separate parts in a radial direction; and
c) the transmission part further includes a separate handle at a proximal end of the hemostasis valve.

In some embodiments, the transmission part further includes a handle at a proximal end of the hemostatic valve.

In some embodiments, a locking mechanism is provided between the handle and the hemostatic valve for keeping an axial distance therebetween.

In some embodiments, the locking mechanism includes a locking groove defined on one of the handle and a proximal end face of the hemostatic valve, and a locking block provided on the other and engaged with the locking groove by rotation.

The present disclosure further provides a guiding device for forming an interventional channel, having opposing proximal and distal ends and including:
an outer sheath being a radially deformable tube and having relative to-be-expanded state and pre-expanded state; and
a driving member having a working portion at its distal end, the working portion having a size so that the working portion is limited to pass through a lumen of the outer sheath in the to-be-expanded state.

The outer sheath and the driving member have the following three states:
a first separated state, in which the outer sheath and the driving member are separated from each other, and the outer sheath is in the to-be-expanded state;
an assembled state, in which the driving member passes through the outer sheath, and two ends of the driving member respectively extend out of the outer sheath, and in which the working portion is located at a distal side of the outer sheath, and the outer sheath is in the to-be-expanded state; and
a second separated state, in which the driving member moves proximally to make the working portion pass through the outer sheath relative to the assembled state, the outer sheath and the driving member are separated from each other, and the outer sheath is in the pre-expanded state.

When switching from the first separated state to the assembled state, the working portion moves from an exterior of the outer sheath to a distal end of the outer sheath.

In some embodiments, when switching from the first separated state to the assembled state, a proximal end of the driving member is inserted into the distal end of the outer sheath until being outside a proximal end of the outer sheath.

In some embodiments, the driving member further includes a handle, and the proximal end of the driving member extends outside the proximal end of the outer sheath and is connected with the handle which has already been arranged at the proximal end of the outer sheath.

In some embodiments, the driving member is formed by separate pieces, at least including a first piece and a second piece arranged from the distal end to the proximal end, and the working portion is provided on the first piece;

When switching from the first separated state to the assembled state, the first piece and the second piece move from the distal end and a proximal end of the outer sheath respectively to be close to and connected with each other.

The present disclosure further provides a guiding device for forming an interventional channel, having opposing proximal and distal ends and including:
an outer sheath being a radially deformable tube;
a hemostatic valve connected to a proximal end of the outer sheath; and
a driving member including a head and a transmission part, the driving member having an initial position and a working direction directed from the initial position toward the proximal end. In the initial position, the head is outside the outer sheath and on a distal side of the outer sheath, and the transmission part is connected with the head and extends inside the outer sheath until being outside the proximal end of the outer sheath through the hemostatic valve. When the driving member moves along the working direction, the head enters the outer sheath and drives a corresponding portion of the outer sheath to radially expand;
In the initial position, a portion of the head radially protruding from the outer sheath forms a working portion, a portion of the transmission part extending out of a proximal end of the hemostatic valve forms an extension section, and the driving member is formed by separate pieces, at least including a first piece and a second piece. The working portion and the extension section are provided on different pieces.

In some embodiments, the extension section has an extension portion protruding radially outward from the outer sheath; the working portion and the extension portion are located on different pieces.

In some embodiments, in assembly, the working portion is always outside the outer sheath, and the two separate pieces move from a distal end of the outer sheath and the proximal end of the outer sheath respectively to be close to each other until being positioned in place to complete the assembly.

In some embodiments, in the initial position, the working portion and the extension portion are fixed relative to the outer sheath in an axial direction.

In some embodiments, the working portion and the extension portion limit the outer sheath and the hemostatic valve in the axial direction, or one of the working portion and the extension portion is engaged with the outer sheath or the hemostatic valve through positioning structures.

In some embodiments, the distal end of the outer sheath has a constraint member for limiting expansion of the outer sheath, and constraint from the constraint member is released before the head enters the outer sheath.

In some embodiments, the transmission part is formed by separate portions in an axial direction, including a distal portion fixed to the head, and a proximal portion connected to the distal portion.

The head and the distal portion are provided on the first piece;

The proximal portion is provided on the second piece.

In some embodiments, the head and the transmission part are separated from each other in an axial direction.

The head is provided on the first piece.

The transmission part is provided on the second piece.

In some embodiments, a limiting structure for limiting movement of the transmission part is provided between a portion of the transmission part extending out of the proximal end of the hemostatic valve and the hemostatic valve.

In some embodiments, the head is connected with the transmission part in a threaded connection.

In some embodiments, the head is formed by separate parts in a radial direction, including:
a central part, a proximal end of which is connected with the transmission part; and
an enlarged part, which is fixedly arranged around an outer periphery of the central part.

The working portion is at least part of the enlarged part and is provided on the first piece; and
the central part is provided on the second piece.

In some embodiments, the central part and the enlarged part are in an interference fit in the radial direction.

In some embodiments, axial limiting structures that are engaged with each other are provided between the central part and the enlarged part.

In some embodiments, the axial limiting structures include an annular groove defined on one of the central part and the enlarged part, and a convex ring arranged on the other and engaged with the annular groove.

In some embodiments, after the head is connected to the transmission part, a proximal end side of the enlarged part abuts against a distal opening of the outer sheath.

In some embodiments, the transmission part further includes a separate handle at the proximal end of the hemostatic valve.

The head and the rest of the transmission part other than the handle are provided on the first piece.

The handle is provided on the second piece.

The present disclosure further provides a guiding device for forming an interventional channel, including:
an outer sheath being a radially deformable tube;
a hemostatic valve connected to a proximal end of the outer sheath; and
a driving member including a head and a transmission part, the driving member having an initial position and a working direction directed from the initial position toward the proximal end. In the initial position, the head is outside the outer sheath and on a distal side of the outer sheath, and the transmission part is connected with the head and extends inside the outer sheath until being outside the proximal end of the outer sheath through the hemostatic valve. When the driving member moves along the working direction, the head enters the outer sheath and drives a corresponding portion of the outer sheath to radially expand;
A portion of the transmission part extending out of the hemostatic valve is a rod-shaped structure, and the rod-shaped structure has a cross-sectional shape allowing the rod-shaped structure to pass through the outer sheath.

In some embodiments, the cross-sectional shape of the rod-shaped structure also allows the rod-shaped structure to pass through the hemostatic valve.

In some embodiments, the portion of the transmission part extending out of the hemostatic valve is provided with a radially outwardly expandable structure.

In some embodiments, the portion of the transmission part extending out of the hemostatic valve is partially recessed.

The present disclosure further provides a guiding device for forming an interventional channel, having opposing proximal and distal ends and including:
an outer sheath being a radially deformable tube;
a hemostatic valve connected to a proximal end of the outer sheath;
a first driving member including a first head and a first transmission part, the first driving member having an initial position and a working direction directed from the initial position toward the proximal end. In the initial position, the first head is outside the outer sheath and on a distal side of the outer sheath, and the first transmission part is connected with the first head and extends inside the outer sheath until being outside the proximal end of the outer sheath through the hemostatic valve. The first head has a fixed shape, and when the first driving member moves along the working direction, the first head enters the outer sheath and drives a corresponding portion of the outer sheath to radially expand; and
a second driving member including a second head and a second transmission part, the second head having a radial size larger than the first head.

In some embodiments, the outer sheath in a to-be-expanded state has an inner diameter of D0, the first head has an outer diameter of D1, and the second head has an outer diameter of D4. D1 is closer to D0 than D4.

In some embodiments, the difference between D4 and D0 is a reference value X, D1−D0=0.05X to 0.4X. For example, D1−D0=0.1X to 0. 2X.

The present disclosure further provides a pre-expansion method for pre-expanding a guiding device, the guiding device including:
an outer sheath being a radially deformable tube and having relative to-be-expanded state and pre-expanded state; and
a driving member having a working portion at its distal end, the working portion having a size so that the working portion is limited to pass through a lumen of the outer sheath in the to-be-expanded state;
the pre-expansion method including steps of:
passing the driving member through the outer sheath in the to-be-expanded state, during which, the working portion moves from an exterior of the outer sheath to a distal end of the outer sheath, and a portion of the driving member on a proximal side of the working portion passes through the outer sheath until being outside a proximal end of the outer sheath; and
moving the driving member proximally to make the working portion pass through the outer sheath so that the outer sheath is in the pre-expanded state.

In some embodiments, when the driving member passes through the outer sheath, the working portion is always on a distal side of the outer sheath.

In some embodiments, the driving member includes a head and a transmission part, the working portion is provided on the head, and the proximal end of the outer sheath is provided with a hemostatic valve;

Passing the driving member through the outer sheath includes inserting a proximal end of the transmission part into the distal end of the outer sheath; and
further moving the driving member proximally relative to the outer sheath until the transmission part moves out of the hemostatic valve.

In some embodiments, the proximal end of the outer sheath is provided with a hemostatic valve, and the driving member includes a head and a transmission part;

Passing the driving member through the outer sheath includes inserting a proximal end of the transmission part into the distal end of the outer sheath; and
further moving the driving member proximally relative to the outer sheath until the transmission part passes through the hemostatic valve and is connected with a handle which has already been arranged at a proximal end of the hemostatic valve.

In some embodiments, the proximal end of the outer sheath is provided with a hemostatic valve, the driving member includes a head and a transmission part, the head is formed by separate parts in a radial direction including a central part and an enlarged part, a proximal end of the central part is connected with the transmission part, the enlarged part is fixedly arranged around an outer periphery of the central part, and the working portion is at least part of the enlarged part;

Passing the driving member through the outer sheath includes inserting a distal end of the transmission part into the proximal end of the outer sheath;
further moving the transmission part distally relative to the outer sheath until the transmission moves out of the outer sheath; and
arranging the enlarged part around the central part from a distal end of the central part.

The present disclosure further provides a pre-expansion method for pre-expanding a guiding device, including:
step S100, providing a driving member and an outer sheath with a hemostatic valve, assembling the driving member into the outer sheath to form any of the guiding devices for forming the interventional channel as mentioned above.
step S200, delivering the guiding device; and
step S300, after reaching a preset site, moving the driving member along the working direction to drive the corresponding portion of the outer sheath to radially expand.

In some embodiments, the driving member in the step S100 is a first driving member, the guiding device further includes a second driving member, and the pre-expansion method further includes:
step S400, moving the first driving member or the second driving member from a proximal end toward a distal end, to perform at least one additional pre-expansion on the outer sheath, and after the additional pre-expansion, proximally withdrawing the first driving member or the second driving member out from the hemostatic valve.

In some embodiments, the method includes performing the additional pre-expansion one or more times, with each additional pre-expansion being performed by the respective second driving members which have the same or different sizes.

In some embodiments, the method includes performing the additional pre-expansion multiple times, and the sizes of the respective second driving members increase in a sequence of the multiple additional pre-expansions performed.

The present disclosure further provides a transcatheter interventional system having opposing proximal and distal ends, including:
any of the above-mentioned guiding devices, the outer sheath in a pre-expanded state forms the interventional channel; and
a delivery system including a catheter assembly, a control handle connected to a proximal end of the catheter assembly, and a prosthetic implant loaded in a distal end of the catheter assembly. The catheter assembly is configured to be delivered through the interventional channel.

After the guiding device of the present disclosure is delivered to a preset site in the human body, the driving member moves along the working direction to expand the outer sheath until the driving member is withdrawn from the human body to form the interventional channel. The operation of withdrawing the driving member from the human body and the expansion operation are completed at the same time to improve efficiency. During the working process of the driving member, the resistance of the outer sheath to the driving member is reduced. Moreover, compared with the existing pushing operation, the pulling operation is less difficult, and has lower requirements on the structural strength of the transmission part.

### BRIEF DESCRIPTION OF DRAWINGS

FIG. 1 is a schematic view of part of an outer sheath in the prior art;

FIG. 2 is a schematic view of a driving member in the outer sheath in FIG. 1 delivered by intervention;

FIG. 3 is a schematic view of a guiding device according to an embodiment of the present disclosure;

FIG. 4 is a partial cross-sectional view of the distal end of the guiding device in FIG. 3;

FIG. 5 is a schematic view of the driving member in FIG. 4 expanding the outer sheath;

FIG. 6 is a schematic view of a cross-section of an outer sheath of a guiding device according to an embodiment of the present disclosure;

FIG. 7 is a partial sectional view of an outer sheath of a guiding device according to another embodiment of the present disclosure;

FIG. 8 is a partial schematic view of a driving member of a guiding device according to an embodiment of the present disclosure;

FIG. 9 is a partial schematic view of a driving member of a guiding device according to another embodiment of the present disclosure;

FIG. 10 is a partial schematic view of a driving member of a guiding device according to another embodiment of the present disclosure;

FIG. 11 is a schematic view of a guiding device in a first separated state according to an embodiment of the present disclosure;

FIG. 12 is a schematic view of the guiding device in FIG. 11 in an assembled state;

FIG. 13 is a schematic view of the guiding device in FIG. 11 in a second separated state;

FIG. 14 is a schematic view of a guiding device in a first separated state according to another embodiment of the present disclosure;

FIG. 15 is a schematic view of the guiding device in FIG. 14 in an assembled state;

FIG. 16 is a schematic view of the guiding device in FIG. 14 in a second separated state;

FIG. 17 is a flowchart of a method for assembling a guiding device according to an embodiment of the present disclosure;

FIG. 18 is a schematic view of a guiding device in a first separated state according to another embodiment of the present disclosure;

FIG. 19 is a schematic view showing the second piece of the guiding device in FIG. 18 being inserted into the outer sheath;

FIG. 20 is a schematic view of the guiding device in FIG. 18 in an assembled state;

FIG. 21 is an enlarged schematic view of part A in FIG. 20;

FIG. 22 is a flowchart of a method for assembling a guiding device according to an embodiment of the present disclosure;

FIG. 23 is a schematic view of a guiding device in a first separated state according to another embodiment of the present disclosure;

FIG. 24 is a schematic view showing the transmission part of the guiding device in FIG. 23 being inserted into the outer sheath;

FIG. 25 is a schematic view of the guiding device in FIG. 23 in an assembled state;

FIG. 26 is a schematic view of a guiding device in a first separated state according to another embodiment of the present disclosure;

FIG. 27 is a schematic view showing the transmission part of the guiding device in FIG. 26 being inserted into the outer sheath;

FIG. 28 is a schematic view of the guiding device in FIG. 26 in an assembled state;

FIG. 29 is a flowchart of a method for assembling a guiding device according to an embodiment of the present disclosure;

FIG. 30 is a partial cross-sectional view of a driving member according to another embodiment of the present disclosure;

FIG. 31 is a partial cross-sectional view of a driving member and an outer sheath according to another embodiment of the present disclosure;

FIG. 32 is a schematic view of a guiding device according to another embodiment of the present disclosure;

FIG. 33 is a schematic view showing that the first driving member in FIG. 32 has completed the pre-expansion operation;

FIG. 34 is a schematic view showing that the second driving member in FIG. 32 starts to perform additional pre-expansion; and

FIG. 35 is a flowchart of a method for forming an interventional channel according to an embodiment of the present disclosure.

### LIST OF THE REFERENCE SIGNS

10, outer sheath; 101, diameter-reduced portion; 11, driving member; 111, bulge;
112, transmission rod; 113, constraint sleeve;
2000, guiding device; 201, proximal end; 202, distal end; 21, outer sheath; 211, diameter-reduced portion; 212, interventional channel; 213, outer film layer; 214, middle layer; 215, inner film layer; 217, constraint member;
22, hemostasis valve; 221, first connection passage; 222, second connection passage;
23, driving member; 231, head; 2311, central part; 2312, enlarged part; 232, transmission part; 233, guide portion; 234, first crown; 235, second crown; 236, main body; 237, working portion; 238, extension portion; 239, lubricating layer; 24, handle;
311, first piece; 312, second piece; 313, extension section;
410, first driving member; 411, first head; 412, first transmission part;
420, second drive member; 421, second head; 422, second transmission part.

### DESCRIPTION OF EMBODIMENTS

The technical solutions according to the embodiments of the present disclosure will be described clearly and fully in combination with the drawings according to the embodiments of the present disclosure. Obviously, the described embodiments are not all embodiments of the present disclosure, but only part of the embodiments of the present disclosure. Based on the disclosed embodiments, all other embodiments obtained by those skilled in the art without creative work fall into the scope of this disclosure.

It should be noted that, when a component is “connected” with another component, it may be directly connected to another component or may be indirectly connected to another component through a further component. When a component is “provided” on another component, it may be directly provided on another component or may be provided on another component through a further component.

Unless otherwise defined, all technical and scientific terms used herein have the same meaning as commonly understood by a person skilled in the art. The terms in the description of the present disclosure are used to describe specific embodiments, and not to limit the present disclosure. The term “and/or” used herein includes one or more of the listed options in any combinations, or the combination of all of the listed options.

In the present disclosure, the terms “first”, “second” and the like are used for descriptive purposes only and are not to be understood as indicating or implying the relative importance or the number or order of the technical features referred. Thus, features defined with “first”, “second” can explicitly or implicitly include one or more of such features. In the description of the present disclosure, “plurality” means at least two, such as two, three, etc., unless explicitly and specifically defined otherwise.

The proximal end herein generally refers to the end adjacent to the operator (such as a doctor), and the distal end refers to the end away from the operator. Along the interventional path, each component has its own opposing distal end and proximal end, and the straight line between the proximal end and the distal end is determined as the axial direction. Accordingly, the radial direction perpendicular to the axial direction and the circumferential direction around the axial direction can be determined.

Outer sheath is usually tubular and configured to form an interventional channel through which a delivery system can reach an operation site in vivo or a simulation site in vitro. For example, in a heart valve replacement, the outer sheath is inserted into the blood vessel to form a surgical access after subcutaneous puncture. Some existing outer sheaths have fixed diameters and need to enclose all implants or devices that need to be interventionally delivered during surgery, so that the outer sheaths have large diameters, and it is difficult for such outer sheaths to adapt to the complicated and tortuous interventional channel.

Some existing outer sheaths are expandable sheaths. For example, the diameter of the outer sheath can be changed by elastic or plastic deformation, so that the outer sheath with a small diameter can enclose all implants or devices that need to be interventionally delivered, to improve the delivery safety and the adaptability of the outer sheath to the tortuous interventional channel.

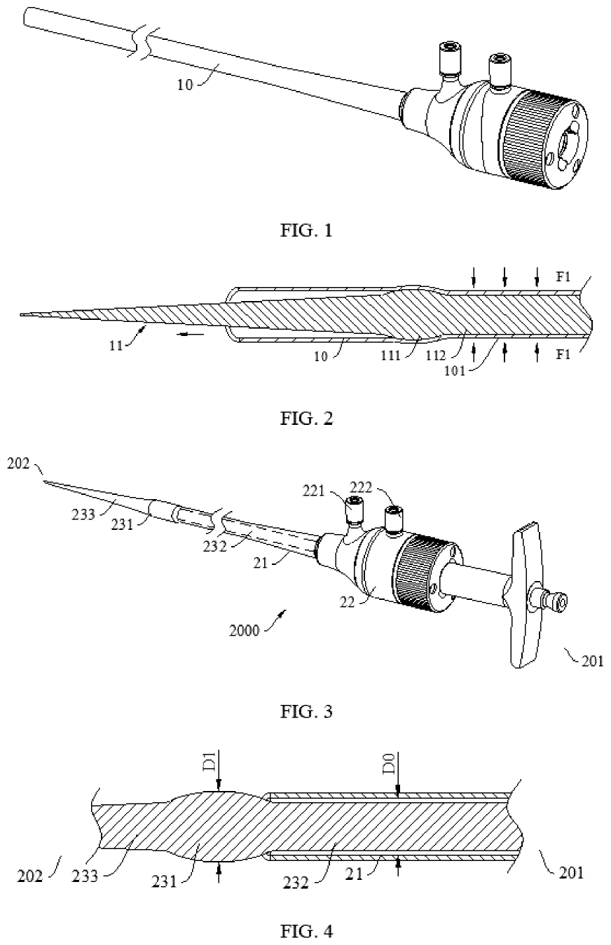

As shown in FIGS. 1 and 2, in use of an existing expandable sheath, the outer sheath 10 to be expanded is first delivered to the human body, and after reaching a preset site, a driving member 11 is pushed into the outer sheath 10. The driving member 11 includes a handle (not shown in the figure) outside the human body, a transmission rod 112 connected to the handle, and a bulge 111 located at the distal end of the transmission rod 112. The proximal end of the outer sheath 10 is controlled by a hemostatic valve. When the driving member 11 is distally pushed to expand the outer sheath 10, the force applied to the outer sheath 10 to stretch the outer sheath 10 toward the distal end makes the portion of the outer sheath 10 on the proximal side of the bulge 111 be radially contracted to form a diameter-reduced portion 101, and the diameter-reduced portion 101 exerts a tightening force F1 shown in FIG. 2 acting on the transmission rod 112 to increase the push resistance to the driving member 11.

Further, as the bulge 111 is far away from the handle, the transmission rod 112 needs to have a certain structural strength, for example, by having a large diameter, to facilitate the force transmission. Due to the diameter-reduced portion 101, the resistance to the transmission rod 112 with a large diameter will be further increased. Moreover, the transmission rod 112 with enhanced structural strength adversely affects the pushing of the driving member 11 in the interventional channel, especially at the turning point.

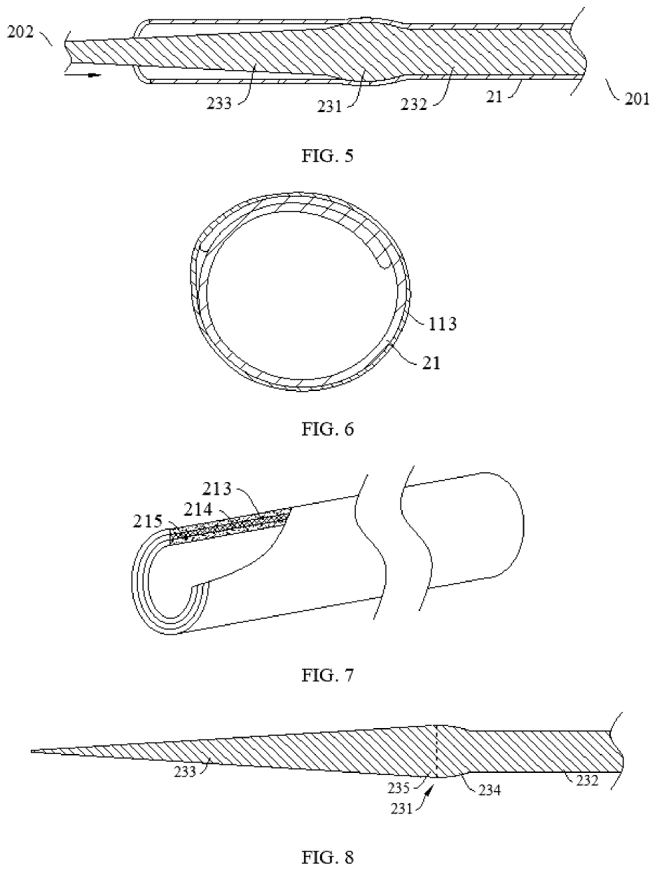

As shown in FIG. 3 to FIG. 7, the present disclosure provides a guiding device 2000 for forming an interventional channel, which has a proximal end 201 and a distal end 202 opposite to each other. The guiding device 2000 includes an outer sheath 21, a hemostatic valve 22 and a driving member 23 as shown in FIG. 11. The outer sheath 21 is a radially deformable tube and has a to-be-expanded state and a pre-expanded state. The hemostatic valve 22 is connected to the proximal end of the outer sheath 21. The driving member 23 includes a head 231 and a transmission part 232. The driving member 23 has an initial position and a working direction directed from the initial position toward the proximal end. In the initial position, the head 231 is outside the outer sheath 21 and located on the distal side of the outer sheath 21, and the transmission part 232 is connected with the head 231 and extends inside the outer sheath 21 until being outside the proximal end of the outer sheath 21 through the hemostatic valve 22. The head 231 has a fixed shape. When the driving member 23 moves along the working direction, the head 231 enters the outer sheath 21 and drives the corresponding portion of the outer sheath 21 to radially expand from the initial diameter (D0 which will be described below) to an expanded diameter, so as to realize pre-expansion.

The head 231 having a fixed shape means that it will not be deformed. Even if the head includes separate parts, it still has a fixed shape after assembly, and the parts will not move relative to each other in use.

The corresponding portion of the outer sheath 21 having the initial diameter is in the to-be-expanded state, the corresponding portion of the outer sheath 21 after being pre-expanded is in the pre-expanded state, and the corresponding portion of the outer sheath 21 being pre-expanded is in the expanded state. The inner diameter of the outer sheath 21 in the to-be-expanded state≤the inner diameter of the outer sheath 21 in the pre-expanded state≤the inner diameter of the outer sheath 21 in the expanded state, depending on the structure and the material properties of the outer sheath 21.

When the outer sheath 21 is radially deformed, it may be uniformly deformed at different parts in the circumferential direction, or only deformed and expanded locally. When the cross section of the outer sheath 21 has an irregular shape, the change trend of the inner diameter corresponds to the change trend of the cross-sectional area.

The cross-sectional area is larger and/or the radial tightening force is smaller in the pre-expanded state of the outer sheath 21 than in the to-be-expanded state.

The smaller the radial tightening force is, the smaller the radial expansion force is required for deformation. After pre-expansion, the frictional resistance to the other components being introduced interventionally will be reduced.

As shown in FIG. 6, in one embodiment, the tube wall of the outer sheath 21 has a coiled structure, and the outer sheath 21 has an expanded state in which the coiled structure of a corresponding portion of the outer sheath 21 is unfolded and a pre-expanded state in which the coiled structure is at least partially restored. In this embodiment, the cross-sectional shape of the outer sheath 21 is close to a circle, and for convenience, the change of the cross-sectional area of the outer sheath is described as the change of the inner diameter.

The tube wall in a preset state is wound more than 360 degrees, with the terminal side being coiled more than 360 degrees in the circumferential direction relative to the start side, and the part wound more than 360 degrees overlapping the part wound within 360 degrees. The outside of the tube wall is covered with a constraint sleeve 113 for limiting the tube wall in the preset state and constraining the tube wall of the outer sheath 21 so that the tube wall cannot be unfolded during interventional delivery, eliminating potential safety hazards during interventional delivery. Further, the constraint sleeve 113 is broken off or torn when the tube wall is expanded, thereby releasing the outer sheath 21 from the constraint of the constraint sleeve 113, so that the tube wall of the outer sheath 21 can be easily unfolded in the subsequent interventional delivery of the delivery system, facilitating the operation.

In this embodiment, releasing the outer sheath 21 from the constraint of the constraint sleeve 113 means that as at least the radial tightening force is reduced. When the constraint sleeve 113 is broken off or torn, the outer sheath 21 would expand and deform at the same time. After the head is withdrawn from the outer sheath 21, if the outer sheath 21 is completely restored to the to-be-expanded state, the inner diameter in the to-be-expanded state=the inner diameter in the pre-expanded state; nevertheless, in some cases, the outer sheath 21 cannot be completely restored, so that the inner diameter of the outer sheath 21 in the to-be-expanded state would be smaller than the inner diameter of the outer sheath 21 in the pre-expanded state.

Before the constraint sleeve 113 is expanded, the outer sheath 21 is in the to-be-expanded state. The constraint sleeve is made of hydrophilic material, which further improves the smoothness of the outer sheath and facilitates the intervening.

As shown in FIG. 7, in one embodiment, the outer sheath 21 is made of elastic material. Specifically, the outer sheath 21 includes an inner film layer 215, a middle layer 214 and an outer film layer 213 in sequence from inside to outside in the radial direction. The inner film layer 215 and the outer film layer 213 are made of synthetic material, which is usually biocompatible material with desired structural strength. In some embodiments, the thickness of the inner film layer 215 ranges from 0.01 mm to 0.5 mm, from 0.02 mm to 0.4 mm, or from 0.03 mm to 0.25 mm. The thickness of the outer film layer 213 ranges from 0.01 mm to 0.5 mm, from 0.02 mm to 0.4 mm, or from 0.03 mm to 0.25 mm.

For example, the inner film layer 215 and the outer film layer 213 can be made of polymer material with elastic modulus of 400 MPa or greater, for example, ultra-high molecular weight polyethylene (UHMWPE) (such as Dyneema), high molecular weight polyethylene (HMWPE) or polyether ether ketone (PEEK).

In order to facilitate the operation of the driving member 23 and the passage of other interventional devices, the inner film layer 215 is made of a material with a low coefficient of friction, such as polytetrafluoroethylene (PTFE), expanded polytetrafluoroethylene (EPTFE), ethylene tetrafluoroethylene (ETFE), nylon, polyethylene, polyether block amides (such as PEBAX), and/or any combination thereof.

In some embodiments, the outer film layer 213 includes an outermost hydrophilic [0151] coating which facilitates the interventional delivery of outer sheath 21.

The middle layer 214 is made of reinforced fiber, which includes a plurality of members or filaments (such as metal wires, synthetic filaments or fibers) braided to adapt to the deformation of the outer sheath 21. The filaments include one or more groups that are braided into each other, and the angle α of each group of filaments relative to the axis of the outer sheath is in the range of 5° to 70°, for example, 10° to 60°, 10° to 50° or 10° to 45°.

In some other embodiments, the outer sheath 21 is not coiled, but has a relatively regular cross-sectional shape in circle or ellipse before or after being pre-expanded. Due to plastic deformation among others, the outer sheath has a larger inner diameter after being pre-expanded.

In other embodiments, the outer sheath 21 itself has a certain elasticity. Although the pre-expanded outer sheath 21 will be elastically restored, it cannot return to the state before the pre-expansion. Therefore, the outer sheath itself would have a reduced radial tightening force and an enlarged inner diameter.

In some embodiments, the reduced radial tightening force may be caused by only microstructural change, such as local peeling between the reinforced fibers and adjacent layers or materials, rearrangement of molecular structures in materials, etc. Although there is no visible diameter change, due to the microstructural change, the outer sheath has a reduced radial tightening force after being pre-expanded. That is, the pre-expanded outer sheath 21 will elastically return to the to-be-expanded state in inner diameter, but the radial tightening force of the outer sheath 21 is significantly reduced.

In one embodiment, the outer film layer 215 includes an elastic layer, and the elastic layer is configured to exert a radially inward force, so that after the driving member is withdrawn from the human body, the outer sheath can at least partially return to the to-be-expanded state.

In the prior art, if the head 231 has a radially deformable structure, the driving member is provided with a mechanism for driving the head 231 to expand inside the transmission part 232 through which the head 231 passes, and accordingly, the handle is provided with a driving mechanism, which makes the structure and operation complicated. In this embodiment, however, the head 231 has a fixed shape, which simplifies the structure and operation. Further, the head 231 with a fixed shape has stronger structural strength, so that the outer sheath 21 can assume the expected pre-expanded state.

Moreover, during expansion, the driving member 23 expands the outer sheath 21 until it is withdrawn from the human body from the proximal end, that is, the pre-expansion operation and the operation of withdrawing from the human body are completed at the same time, to improve the efficiency. The expansion operation of the guiding device according to this embodiment includes pulling the driving member outside the human body. Compared with the existing operation of distally pushing the driving member into the human body, the requirement for the structural strength of the transmission part 232 is lowered, and the difficulty of delivering the guiding device and the expansion operation in the human body is reduced.

Further, during pre-expansion, various resistances such as frictional resistance will be generated between the head and the outer sheath. However, when the pre-expansion ends, the resistance disappears instantly, while the driving member will continue moving quickly in the previous movement direction due to the inertia. For example, in the existing pre-expansion operation of distally pushing the driving member, when the head moves out of the distal end of the outer sheath, it will continue moving toward the distal side uncontrollably due to inertia, or the operation handle will hit the hemostatic valve to generate vibration, resulting in tissue damage. However, the pre-expansion operation of pulling the driving member proximally herein removes such potential safety hazard. For convenience, the following embodiments accompanying drawings will be described with the outer sheath 21 being exemplified as a round tube.

As shown in FIGS. 8 to 10, in one embodiment, the head 231 includes a first crown 234 located at its proximal end, and the first crown 234 is gradually enlarged from the proximal end toward the distal end. In the initial position, the proximal side of the first crown 234 abuts against the distal opening of the outer sheath 21, thereby preventing the opening of the outer sheath from damaging the tissue during the interventional delivery and thus reducing potential safety hazards. Specifically, the outer peripheral surface of the first crown 234 is a chamfered surface or a tapered surface, to facilitate the distal end of the outer sheath 21 to turn outwardly during the pre-expansion so that the head 231 can enter the outer sheath 21. In a preferred embodiment, the junction of the first crown 234 and the transmission part 232 has a smooth surface.

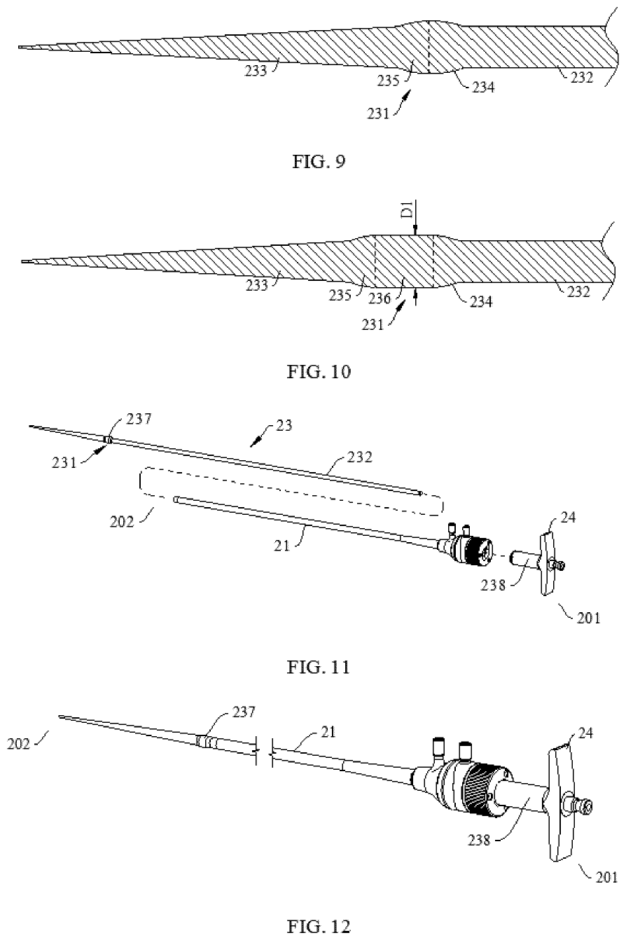

In one embodiment, the head 231 is tapered from the largest diameter of the first crown 234 toward the distal end, to provide a guiding effect during the delivery in vivo or in a simulated environment in vitro. The tapering form can be a continuously tapering form as shown in FIG. 8, or stepwise tapering form as shown in FIG. 9. For example, the head 231 further includes a second crown 235 at the distal side, and the second crown 235 tapers from the largest diameter of the first crown 234 toward the distal end.

In one embodiment, the distal end of the second crown 235 is further connected with a guide portion 233, and the guide portion 233 also tapers toward the distal end. In the case of continuously tapering form, a visible division is not presented between the second crown 235 and the guide portion 233, and the second crown 235 and the guide portion 233 are tapered on the same slope. In the case of stepwise tapering form, the second crown 235 has a greater inclination relative to the axis of the head 231 than the guide portion 233 and a visible division is presented between them. Referring to FIG. 4 again, the radial dimension (i.e., the maximum radial dimension) of the head 231 is D1, the inner diameter of the outer sheath 21 in the to-be-expanded state is D0, and D1 is greater than D0.

In some embodiments, the head 231 formed by the first crown 234 and the second crown 235 is generally spherical or ellipsoidal. As shown in FIG. 10, in another embodiment, the head 231 further includes a main body 236 extending in an equal diameter between the first crown 234 and the second crown 235. Among the main body 236, the first crown 234 and the second crown 235, the main body 236 has the largest diameter D1. The junctions between the first crown 234, the second crown 235 and the main body portion 236 are chamfered. For the above-mentioned different embodiments, the intersections between the first crown 234, the second crown 235, and the main body portion 236 are shown as dotted lines in the figures.

The transmission part 232 can be a solid or hollow rod, and the proximal end thereof is provided with another structure to be hold for the pre-expansion operation. The cross section of the transmission part 232 is not strictly limited, but a circle or an ellipse is preferred for providing a smooth outer surface.

The hemostatic valve 22 can use the existing hemostatic valve, including a housing and a sealing element inside the housing. The sealing element can be an elastic element or a deformable element driven by fluid. When the catheter assembly of the delivery system passes through the hemostatic valve, blood leakage is prevented.

The hemostatic valve 22 is provided with a first connection passage 221 for injecting fluid to drive the sealing element and a second connection passage 222 for gas discharging.

The hemostatic valve has a first channel therein on which the sealing element acts to close or open the same. The driving member 23 passes through the first channel and the interventional channel in the outer sheath 21. As the driving member 23 is withdrawn from the proximal end of the hemostatic valve, the first channel should at least allow the head 231 to pass through.

As shown in FIGS. 11 to 13, the present disclosure further provides a guiding device for forming an interventional channel, which has opposing proximal and distal ends. The guiding device includes an outer sheath 21 and a driving member 23. The outer sheath 21 refers to the above-mentioned embodiments. The distal end of the driving member 23 is provided with a working portion 237, and the working portion 237 has a size so that the working portion 237 is limited to pass through the lumen of the outer sheath 21 in the to-be-expanded state. Therefore, the pre-expansion operation cannot be performed before the guiding device reaches the operation site. Only after the guiding device reaches the operation site, the pre-expansion operation can be performed by applying force on the driving member, so that the pre-expansion operation is controllable and misoperation is prevented.

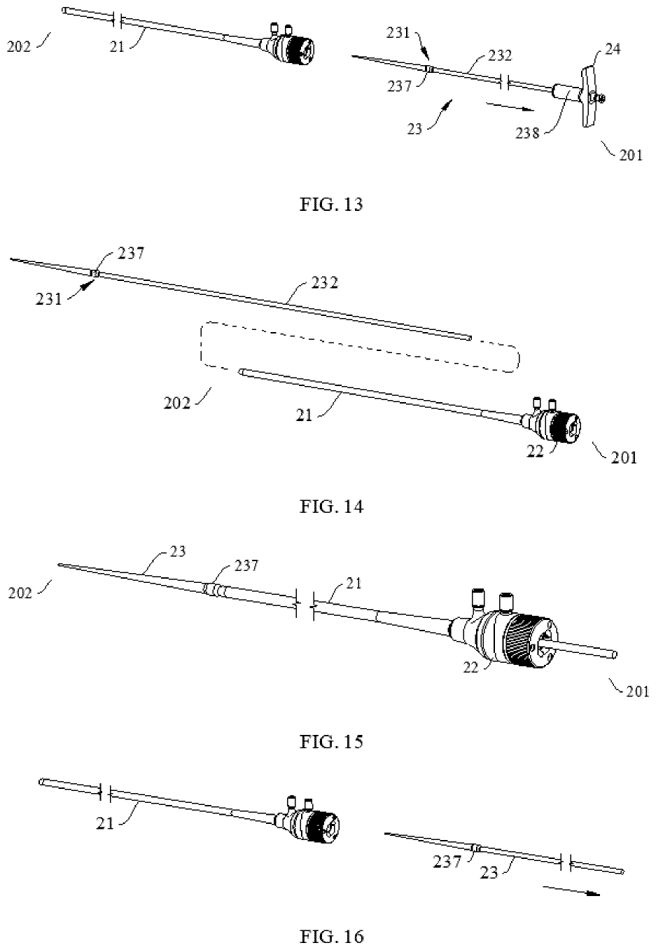

The outer sheath 21 and the driving member 23 have the following three states with each other:
a first separated state as shown in FIG. 11, in which the outer sheath 21 and the driving member 23 are separated from each other, and the outer sheath 21 is in a to-be-expanded state;
an assembled state as shown in FIG. 12, in which the driving member 23 passes through the outer sheath 21, and the two ends thereof respectively extend out of the outer sheath 21, and in which the working portion 237 is located on the distal side of the outer sheath, and the outer sheath 21 is in the to-be-expanded state; when switching from the first separated state to the assembled state, the working portion 237 moves from the exterior of the outer sheath to the distal end of the outer sheath 21, that is, the working portion 237 doesn't move to the distal end from the interior of the outer sheath 21; and
a second separated state as shown in FIG. 13, in which the driving member 23 moves proximally so that the working portion 237 passes through the outer sheath 21 until being outside the outer sheath 21, the driving member 23 and the outer sheath 21 are separated from each other relative to the assembled state, and the outer sheath 21 is in a pre-expanded state.

Due to the size of the working portion 237, the working portion 237 cannot pass through the outer sheath 21 in the to-be-expanded state. When performing pre-expansion, since the outer sheath is radially deformable, the working portion 237 is allowed to pass through the deformable outer sheath. That is, the pre-expansion is realized based on the deformation of the outer sheath.

The working portion 237 can be a separate component, or a portion of the driving member 23. For example, from the perspective of the axial direction, the portion of the distal end of the driving member outside the inner edge of the outer sheath corresponds to the working portion 237.

The shape of the working portion is not strictly limited, as long as it is convex in the radial direction to a certain degree, so that it can be blocked by the outer sheath in the to-be-expanded state. In some cases, the working portion 237 can be annular, so that the stress distribution on the working portion 237 and the outer sheath with a circular cross section during the pre-expansion can be more reasonable.

When the guiding device is delivered into the human body and reaches the operation site or for simulation training in vitro in an assembled state, the pre-expansion is performed manually until the guiding device is transformed into the second separated state, to form the interventional channel. The process from the to-be-expanded state to the pre-expand state can refer to the above-mentioned embodiments, in which the driving member 23 moves along the working direction, and the working portion 237 enters the outer sheath 21 and finally moves out from the proximal end of the outer sheath.

Depending on the size relationship between the working portion 237 and the outer sheath 21 and the configuration of the driving member 23 (in one piece or separate pieces), the outer sheath 21 and the driving member 23 are assembled to each other in the assembled state from the first separated state as follows:

As shown in FIGS. 14 to 16, in one embodiment, the driving member 23 is formed in one piece, and the portion of the transmission part 232 extending out of the hemostasis valve 22 is a rod-shaped structure, and the cross-sectional shape of the rod-shaped structure allows the rod-shaped structure to pass through the outer sheath 21. In assembly, the proximal end of the driving member 23 is inserted from the distal end of the outer sheath 21 until it moves out from the proximal end of the hemostatic valve 22 (that is, the cross-sectional shape of the rod-shaped structure also allows the rod-shaped structure to pass through the hemostatic valve). The transmission part 232 does not pre-expand the outer sheath 21 when passing through the outer sheath 21. The transmission part 232 extends in an equal diameter. Alternatively, in some embodiments, the portion of the transmission part 232 extending out of the proximal end of the hemostatic valve has a shaped structure, such as a local recess, a local protrusion or a radially expandable and controllable structure by deformation or expansion or the like, to facilitate the operator to hold.

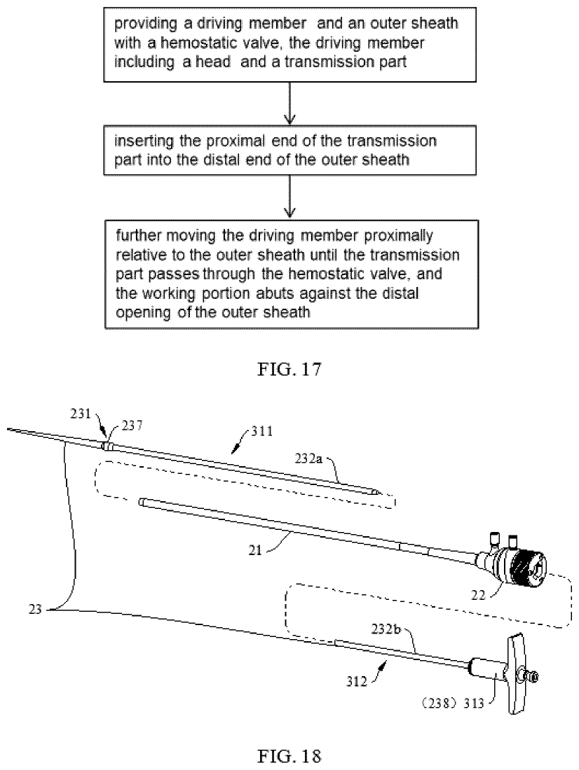

As shown in FIG. 17, according to the guiding device of this embodiment, a method for assembling the guiding device is provided, including:
S100, providing a driving member 23 and an outer sheath 21 with a hemostatic valve 22; the driving member 23 includes a head 231 and a transmission part 232, the proximal end of the transmission part is a rod-shaped structure, and the cross-sectional shape of the rod-shaped structure allows the rod-shaped structure to pass through the outer sheath 21;
S200, inserting the proximal end of the transmission part 232 into the distal end of the outer sheath 21; and
S300, further moving the driving member 23 proximally relative to the outer sheath 21 until the transmission part 232 passes through the hemostatic valve 22, and the working portion 237 abuts against the distal opening of the outer sheath 21.

The portion of the transmission part extending out of the proximal end of the hemostatic valve can be configured to be deformable, and after being deformed, it is blocked by the hemostatic valve, so that the working portion and the portion of the transmission part extending out of the proximal end of the hemostatic valve can limit the outer sheath and the hemostatic valve in the axial direction, to keep the axial relative positional relationship of the components of the guiding device fixed.

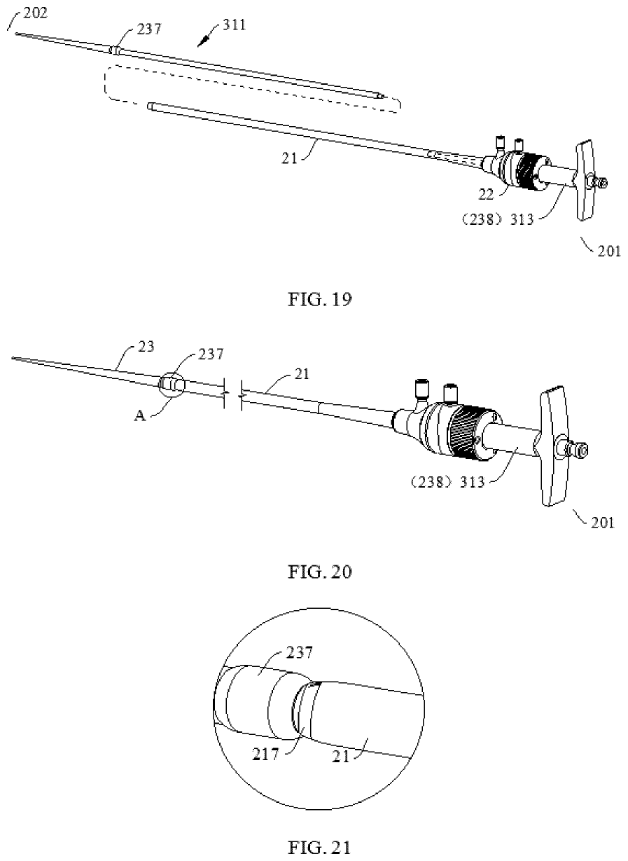

As shown in FIGS. 18 to 20, in one embodiment, the outer sheath 21 is a radially deformable tube, the hemostatic valve 22 is connected to the proximal end of the outer sheath 21, and the driving member 23 includes a head 231 and a transmission part 232. In the initial position, the portion of the head 231 radially protruding outward from the outer sheath 21 forms a working portion 237, and the portion of the transmission part 232 extending out of the proximal end of the hemostatic valve forms an extension section 313. The driving member 23 is formed by separate pieces, including at least two separate pieces, for example, a first piece 311 and a second piece 312, and the working portion 237 and the extension section 313 are formed on different pieces.

The first piece and the second piece can be respectively formed in one piece or separate pieces. The driving member 23 is formed by separate pieces, which, however, have already been assembled together at the initial position, and the assembly can be done in the manufacture process, or in the practical procedure by the doctor.

The two separate pieces are respectively the first piece 311 with the working portion 237, and the second piece 312 with the extension section. The second piece 312 is inserted from the proximal end of the hemostatic valve 22, and connected with the first piece 311 on the distal side. The connection may be proximal to the hemostatic valve, inside the outer sheath, or distal to the outer sheath.

The reason why the driving member 23 includes separate pieces is that the shape of the head 231 is fixed. If the driving member 23 is inserted from the proximal end of the outer sheath, pre-expansion would occur, which will adversely affect the diameter or the axial pushing performance of the outer sheath and thus the interventional delivery of the outer sheath in vivo. However, in this embodiment, as the driving member 23 is formed by separate pieces, the first piece 311 and the second piece 312 can be inserted from the distal end of the outer sheath 21 and the proximal end of the hemostatic valve 22 respectively and connected with each other during assembly, which facilitates the assembly and eliminates the adverse effects mentioned above. The first piece 311 includes the head and a portion of the transmission part, and the second piece 312 includes the other portion of the transmission part and the extension section.

The connection of the separate pieces can be located between the extension section and the transmission part 232, or between the transmission part 232 and the head 231, or on the transmission part 232 as shown in the figure (in the later case, after assembly, the connection of the separate pieces is located inside the outer sheath 21).

The extension section 313 may be configured as a handle as shown in the figure, but is not limited to a handle.

Referring again to FIGS. 11 to 13 and FIGS. 18 to 20, in one embodiment, in the initial position, in addition that the working portion 237 formed on the head 231 radially protrudes outward from the outer sheath 21, an extension portion 238 provided on the portion of the transmission part 232 extending out of the proximal end of the hemostatic valve also radially protrudes outward from the outer sheath 21. The driving member 23 is formed by separate pieces, including at least two separate pieces, and the working portion 237 and the extension portion 238 are formed on different pieces.

The extension portion 238 is located on the second piece 312 and is used for the operator to hold for the pre-expansion operation, which may be part or the entire of the extension section of the above-mentioned embodiments. In order to facilitate the operator to hold and the force application by the operator, the radial size of the extension portion 238 hardly allows the extension portion 238 to pass through the outer sheath 21 and the hemostatic valve. During assembly, the working portion 237 is always outside the outer sheath 21, and the two separate pieces approach each other from the distal side of the outer sheath and the proximal side of the outer sheath respectively until they are respectively positioned in place to complete the assembly. The working portion 237 and the extension portion 238 can each have a fixed shape. The benefits for the working portion 237 having a fixed shape can be referred to the above-mentioned embodiments, which will not be repeated here. The extension portion 238 having a fixed shape facilitates the operator to hold and the force application by the operator, omitting any complicated expandable structure.

The first piece 311 and the second piece 312 can be connected with each other in a snap-fit or threaded connection. For example, the connection can be located between the transmission part 232 and the head 231, that is, the head 231 and the transmission part 232 are separate in the axial direction, with the head 231 being provided on the first piece 311 and the transmission part 232 being provided on the second piece 312. During assembly, the transmission part 232 is inserted from the proximal end to the distal end of the outer sheath 21 and connected with the head 231.

As shown in FIG. 18, the connecting between the first piece 311 and the second piece 312 can be alternatively located on the transmission part 232 and inside the outer sheath 21. That is, the transmission part 232 includes separate portions in the axial direction, including a distal portion 232a fixed to the head 231 and a proximal portion 232b connected to the distal portion 232a.

The head 231 and the distal portion 232a are provided on the first piece 311, and the proximal portion 232b is provided on the second piece 312. In assembly, the proximal portion 232b is inserted from the proximal end of the outer sheath toward the distal end, and the distal portion 232a is inserted from the distal end of the outer sheath toward the proximal end and connected with the proximal portion 232b inside the outer sheath 21.

In one embodiment, the working portion 237 and the extension portion 238 limit the outer sheath 21 and the hemostatic valve 22 in the axial direction. Alternatively, one of the working portion 237 and the extension portion 238 is engaged with the outer sheath 21 or the hemostatic valve 22 through positioning structures. During the interventional delivery of the driving member, the relative positional relationship between the driving member and the outer sheath is fixed and the components in assembly are positioned in place. During pre-expansion, the positioning structures are disengaged to allow the driving member to move along the working direction. The outer sheath 21 and the hemostatic valve 22 can be limited in the axial direction through the working portion 237 and the extension portion 238 which clamp the outer sheath 21 and the hemostatic valve 22 in the axial direction.

For example, the positioning structures can be provided between the extension portion 238 and the proximal end of the hemostasis valve to keep the relative positional relationship between them fixed. Specifically, the extension portion 238 and the hemostasis valve are relatively fixed at least in the axial direction through the positioning structures, so that after the second piece 311 is connected with the first piece 312, the proximal end of the working portion 237 can attach with or abut against the outer sheath 21 to close the distal opening of the outer sheath 21. The two protruding portions (the working portion and the extension portion) limiting the outer sheath 21 and the hemostasis valve 22 in the axial direction functions similar to the positioning structures.

In one embodiment, the transmission part 232 further includes a separate handle 24 at the proximal end of the hemostasis valve 22, that is, the rod-shaped structure and the handle 24 of the transmission part 232 are separate from each other. The handle 24 can be configured as the extension portion mentioned in the above embodiment. The handle 24 and the proximal end of the hemostatic valve are provided with a locking mechanism for fixing the axial distance between the two, specifically for fixing the relative positional relationship between the driving member and the outer sheath during the interventional delivery of the driving member. During pre-expansion, unlocking the locking mechanism allows the handle to axially move relative to the hemostatic valve. The handle can use an existing handle.

The locking mechanism includes a locking groove defined on one of the handle and the proximal side of the hemostasis valve, and a locking block provided on the other and engaged with the locking groove by rotation.

In another embodiment, the handle and the hemostatic valve are threadedly connected, and the locking mechanism includes the threads respectively provided by the handle and the hemostatic valve, which are engaged with each other. Specifically, the handle is provided with a male thread that engages with the hemostatic valve and a female thread that engages with the rest of the transmission part.

As shown in FIG. 21, in one embodiment, the distal end of the outer sheath 21 is provided with a constraint member 217 for limiting the expansion of the outer sheath 21. The constraint from the constraint member 217 is released before the head enters the outer sheath 21. During assembly, the working portion 237 of the first piece can be positioned against the constraint member 217 so as to be positioned at the distal end, and then the first piece is connected with the second piece. Further, the head 231 will not accidentally enter the outer sheath 21, which is convenient for transportation. Of course, before delivery, the constraint member 217 needs to be removed so that the distal end of the outer sheath 21 can return to its preset shape for the subsequent pre-expansion. The constraint member 217 can be a ring, a bundle, or the like.

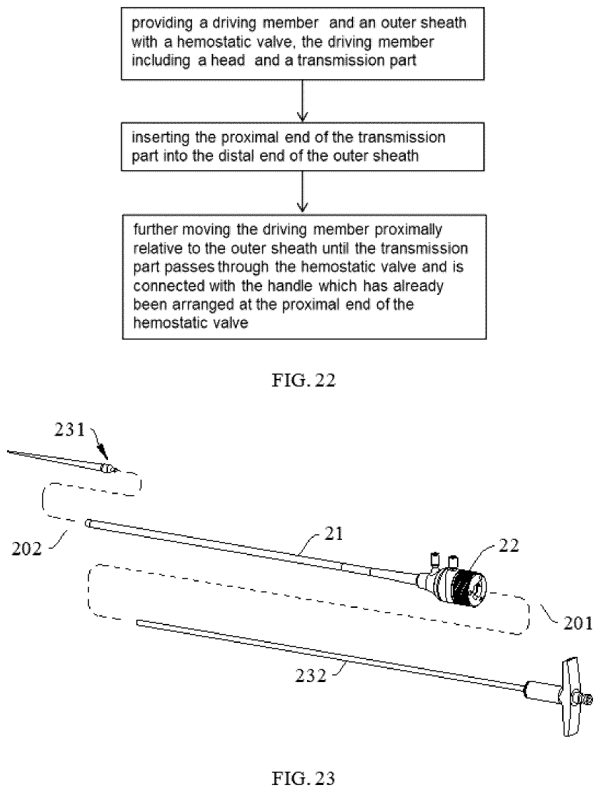

As shown in FIG. 22, according to the above-mentioned guiding device with a handle, a method for assembling the guiding device is provided, including:
S100, providing a driving member 23 and an outer sheath 21 with a hemostatic valve 22; the driving member 23 includes a head 231 and a transmission part 232;
S200, inserting the proximal end of the transmission part 232 into the distal end of the outer sheath 21; and
S300, further moving the driving member proximally relative to the outer sheath 21 until the transmission part 232 passes through the hemostatic valve 22 and is connected with the handle 24 which has already been arranged at the proximal end of the hemostatic valve 22. Specifically, the handle 24 is screwed to the hemostasis valve 22 first and locked by the locking mechanism, and then the transmission part is inserted from the distal end of the outer sheath until the proximal end of the transmission part abuts against the handle, and the distal portion of the transmission part outside the outer sheath (i.e., the head) is rotated to connect the proximal end of the transmission part with the handle. Before the pre-expansion operation, the locking mechanism is unlocked (i.e., the handle 24 is rotated to release the connection thereof with the hemostatic valve 22).

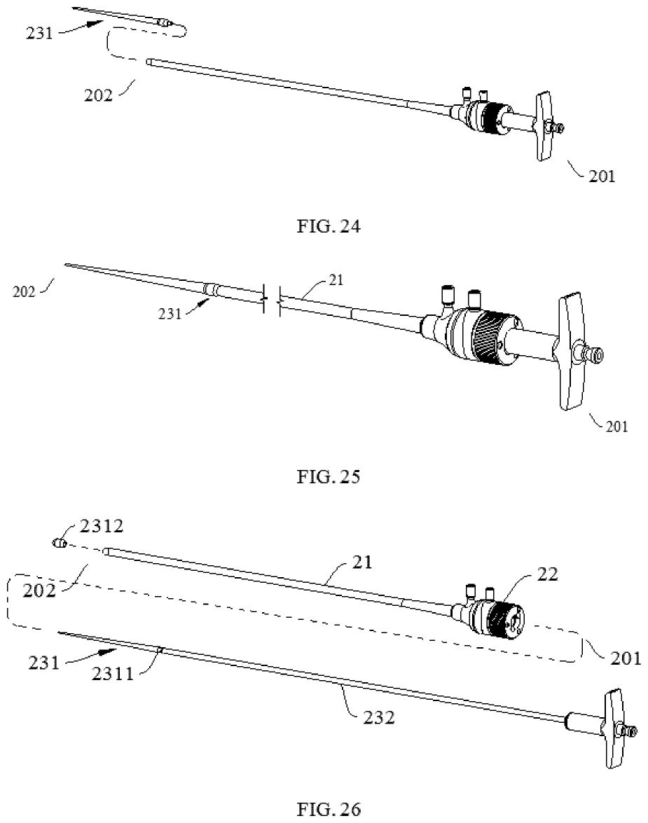

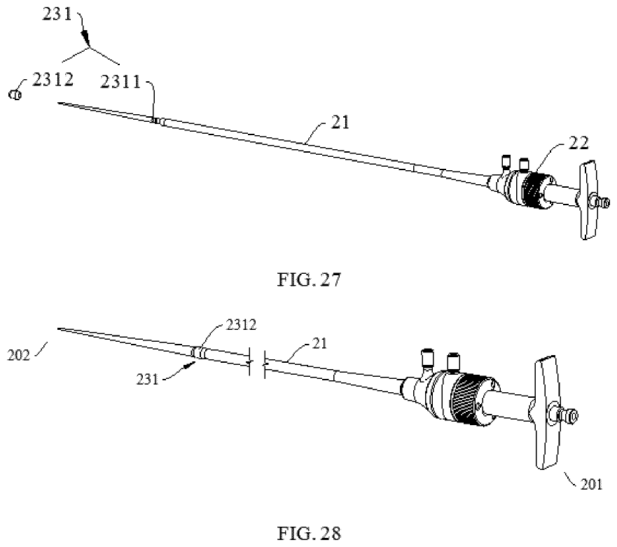

As shown in FIG. 23 to FIG. 28, in another embodiment, the guiding device for forming the interventional channel has opposing proximal and distal ends, and the guiding device includes:
an outer sheath 21, a hemostatic valve 22 and a driving member 23. The outer sheath 21 and the hemostatic valve 22 can refer to the above-mentioned embodiments. The driving member 23 includes a head 231 and a transmission part 232 which are separate from each other. The specific assembly method is as follows:

For example, as shown in FIGS. 23 to 25, the head 231 and the transmission part 232 are separate from each other in the axial direction. The two are axially assembled and connected with each other in a threaded connection or snap fit. A limiting structure for limiting the movement of the transmission part 232 is provided between the portion of the transmission part 232 extending out of the proximal end of the hemostatic valve and the hemostatic valve 22. The limiting structure can use the same structure as the above-mentioned locking mechanism. In assembly, the transmission part 232 is first inserted from the proximal end of the hemostatic valve until being locked by the limiting structure, and thus be fixed relative to the outer sheath 21, and then the head 231 is connected with the transmission part 232 at the distal end of the outer sheath 21.

The head 231 and the transmission part 232 that are separate from each other in the axial direction can be assembled to each other using the above-mentioned assembling method for the driving member with a handle.

For another example, as shown in FIGS. 26 to 28, the head 231 itself includes separate parts in the radial direction. Specifically, the head 231 includes a central part 2311 and an enlarged part 2312. The central part 2311 is a solid or hollow rod, and the proximal end of the central part is connected to the transmission part 232, or the central part and the transmission part 232 are formed in one piece.

The size of the enlarged part 2312 is designed so that at least part of the enlarged part 2312 cannot pass through the outer sheath 21 in the to-be-expanded state. When performing pre-expansion, since the outer sheath is radially deformable, the enlarged part 2312 is allowed to pass through the deformable outer sheath. That is, the pre-expansion is realized based on the deformation of the outer sheath. Specifically, from the perspective of the axial direction, the portion outside the outer edge of the central part 2311 is the enlarged part 2312.

In one embodiment, the enlarged part 2312 is fixedly arranged around the outer periphery of the central part 2311. In assembly, the transmission part 232 with the central part 2311 is inserted from the proximal end of the hemostatic valve until being outside the distal end of the outer sheath and the transmission part is relatively fixed to the hemostatic valve through the limiting structure, and then the enlarged part 2312 is arranged around the central part 2311.

At least part of the enlarged part can be considered as the working portion mentioned above. In the case where the driving member includes separate pieces, the enlarged part is provided on the first piece, and the central part is provided on the second piece.

The enlarged part 2311 is hollow and formed by a ring or two half-rings in a snap-fit with each other. The enlarged part 2311 and the central part can be assembled together using the following method.

For example, the enlarged part 2311 can be a ring in one piece, and the central part 2311 and the enlarged part 2312 can be in an interference fit in the radial direction so that when the transmission part 232 is fixed relative to the outer sheath 21, the central part 2311 can be installed from the distal end around the central part 2311.

For another example, axial limiting structures that are engaged with each other are provided between the central part 2311 and the enlarged part 2312. The axial limiting structures include an annular groove defined on one of the central part and the enlarged part, and a convex ring arranged on the other and engaged with the annular groove. After the head 231 is connected with the transmission part 232, the proximal side of the enlarged part 2312 abuts against the distal opening of the outer sheath 21.

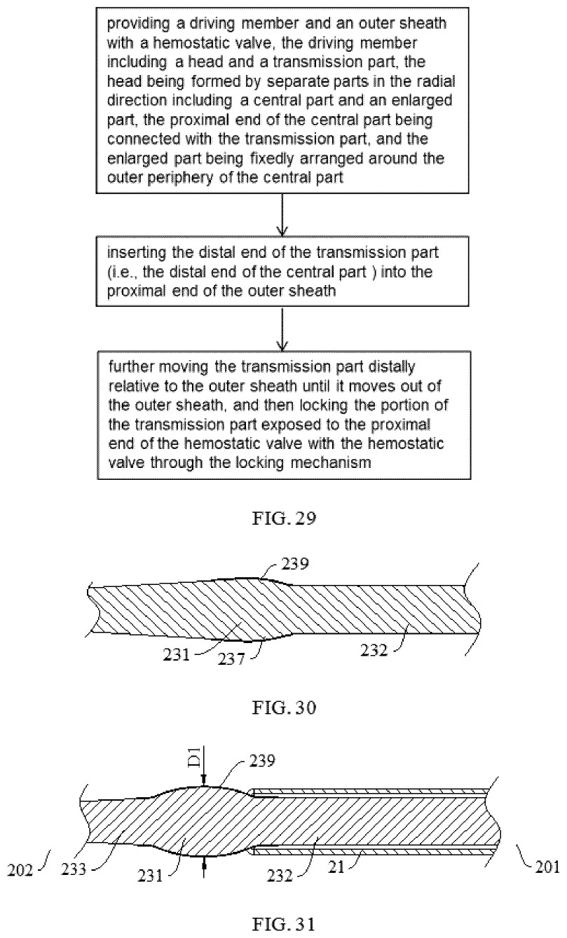

As shown in FIG. 29, according to the guiding device with the head and the transmission part in separate pieces, a method for assembling the guiding device is provided, including:
S100, providing a driving member 23 and an outer sheath 21 with a hemostatic valve 22; the driving member 23 includes a head 231 and a transmission part 232, the head 231 is formed by separate parts in the radial direction including a central part 2311 and an enlarged part 2312, the proximal end of the central part 2311 is connected with the transmission part 232, and the enlarged part 2312 is fixedly arranged around the outer periphery of the central part 2311;
S200, inserting the distal end of the transmission part 232 (i.e., the distal end of the central part) into the proximal end of the outer sheath 21;
S300, further moving the transmission part 232 distally relative to the outer sheath 21 until it moves out of the outer sheath 21, and then locking the portion of the transmission part 232 exposed to the proximal end of the hemostatic valve with the hemostatic valve through the locking mechanism; and
S400, arranging the enlarged part 2312 around the central part 2311 from the distal end of the central part 2311 until the enlarged part 2312 abuts against the distal opening of the outer sheath.

As shown in FIG. 30 and FIG. 31, in one embodiment, the guiding device includes a lubricating layer 239 (shown in thickened lines in the figure) disposed outside the head 231. The lubricating layer 239 at least covers the radially enlarged portion of the head 231, i.e., the above-mentioned working portion 237, to reduce the frictional resistance with the outer sheath 21 during pre-expansion, and reduce the operation influence due to inertia. In this case, the above-mentioned D1 corresponds to the outer diameter of the lubricating layer 239 as shown in FIG. 31. In a specific embodiment, in the assembled state, the proximal end of the lubricating layer 239 extends to the contact portion of the head 231 with the outer sheath 21, so as to guide the head to smoothly enter the outer sheath at the beginning of the pre-expansion.

The lubricating layer 239 can be formed by:
a lubricating tube which is arranged around the head 231 and then attached to the outer periphery of the head 231 by elasticity or heat shrinkage; or
a coating, such as hydrophilic coating, etc.

The lubricating layer can be preset on the head, or set after the driving member is in the assembled state.

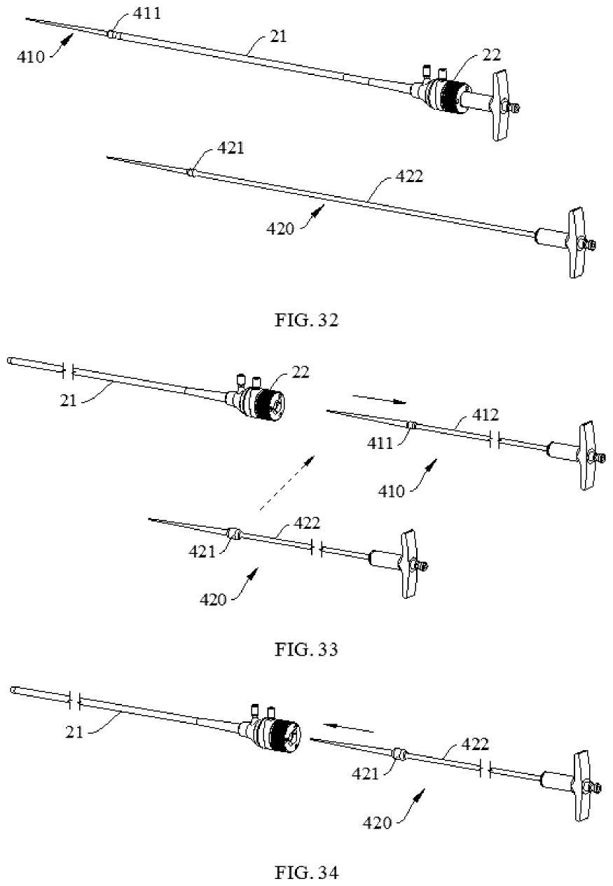

As shown in FIG. 32 to FIG. 34, the present disclosure further provides a guiding device, which has opposing proximal and distal ends. The guiding device includes an outer sheath 21, a hemostatic valve 22, a first driving member 410 and a second driving member 420. The outer sheath 21 and the hemostatic valve 22 refer to the above-mentioned embodiments. The first driving member 410 includes a first head 411 and a first transmission part 412, which refer to the above-mentioned embodiments.

The second driving member 420 includes a second head 421 and a second transmission part 422. The radial dimension D4 of the second head 421 is greater than the radial dimension D1 of the first head 411. After the first driving member 410 pre-expands the outer sheath, the second driving member 420 can enter the outer sheath from the proximal end of the hemostatic valve and drive the corresponding portion of the outer sheath to further expand in the radial direction (hereinafter, such process is described as additional pre-expansion). The figure shows only one second driving member 420. In FIG. 32, the first driving member 410 and the outer sheath 21 have already been assembled together. The second driving member 420 is initially independent from the outer sheath 21, and then pre-expands the outer sheath 21 after the first driving member 410 pre-expands the outer sheath 21.

The second driving member 420 can use the existing technique. After the pre-expansion operation of the first driving member 410 is completed (that is, the first driving member 410 is withdrawn out from the proximal end of the hemostatic valve), the second driving member 420 is inserted from the proximal end of the hemostatic valve to further pre-expands the outer sheath 21 through the second head 421 to further reduce the radial tightening force or enlarge the inner diameter, facilitating the subsequent interventional delivery of the delivery system. The first pre-expansion reduces the resistance between the outer sheath and the second driving member during the additional pre-expansion and the safety hazard caused by inertia.

The additional pre-expansion can be operated in a full stroke, in which case the second head 421 of the second driving member 420 moves out from the distal end of the outer sheath, or in a partial stroke, in which case the second head 421 of the second driving member 420 does not move out from the distal end of the outer sheath. In any case, the second driving member 420 is finally withdrawn from the human body.

In one embodiment, a plurality of second driving members 420 are provided, and in the order of the additional pre-expansions, the second head of the subsequent second driving member should be larger than the second head of the previous second driving member. The previous additional pre-expansion reduces the resistance between the outer sheath and the second driving member during subsequent additional pre-expansion.

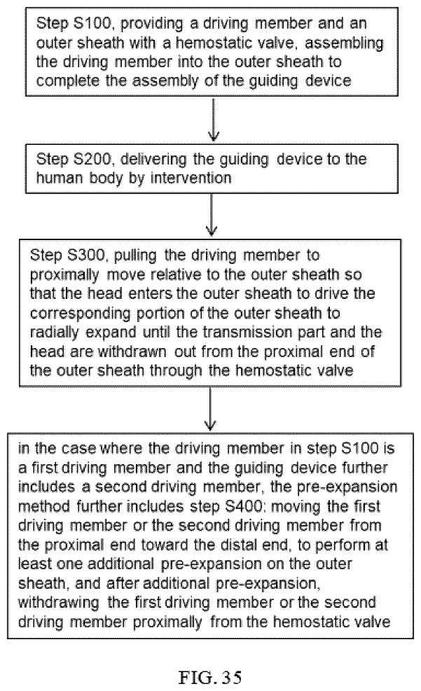

As shown in FIG. 35, an embodiment of the present disclosure further provides a method for forming an interventional channel, including:
step S100, providing a driving member and an outer sheath with a hemostatic valve which can be the driving member and the outer sheath mentioned above; assembling the driving member into the outer sheath using the above-mentioned assembly method for the separate pieces, to complete the assembly of the guiding device;
Step S200, delivering the guiding device to the human body by intervention, or simulating the interventional delivery of the guiding device outside the human body; and
Step S300, after reaching a preset site, moving the driving member along the working direction to transform the outer sheath into the pre-expanded state.

Specifically, the handle is pulled proximally relative to the hemostatic valve after the locking mechanism (if there is) between them is unlocked. The handle drives the head through the transmission part to enter the outer sheath from the distal end of the outer sheath. The portion of the outer sheath in contact with the head is deformed, at least at the moment in contact, by the radial support of the moving head, to realize pre-expansion.

After the entire driving member is withdrawn from the proximal end of the hemostatic valve, the pre-expanded outer sheath forms a temporary interventional channel for delivery of other interventional components.

In the case where the driving member in step S100 is the first driving member 410, the pre-expansion method further includes step S400: moving the first driving member 410 or the second driving member 420 from the proximal end toward the distal end, to perform at least one additional pre-expansion on the outer sheath 21.

After additional pre-expansion, the first driving member 410 or the second driving member 420 is proximally withdrawn from the hemostatic valve 22.

The additional pre-expansion can be performed multiple times, and each additional pre-expansion is performed by the second driving member 420. The sizes of the second driving members (the outer diameters of the second heads) are the same or different. For example, the sizes of the second heads increase sequentially. In the various additional pre-expansions, the maximum outer diameter of the second heads of the second driving members 420 is D4. Alternatively, after the first pre-expansion, the additional pre-expansion can be performed by the first driving member, or a second driving member with a larger head.

The stroke for the additional pre-expansion is not strictly limited, which may be a full stroke or a partial stroke relative to the outer sheath. In an additional pre-expansion, the driving member enters the outer sheath from the proximal end of the hemostatic valve and is pushed distally, and then moves proximally until being withdrawn to the outside of the human body, or withdrawn to the outside of the human body after moving back and forth in the outer sheath.

The second driving member can use an existing driving member, and the size of the second head at its distal end should be greater than the inner diameter of the outer sheath. For example, the inner diameter of the outer sheath in the to-be-expanded state is D0, the first driving member can be used first for the pre-expansion, the outer diameter of the first head of the first driving member (in case with the lubricating layer, the outer diameter of the lubricating layer) is D1, and after pre-expansion, the inner diameter of the outer sheath is increased to D3 or the radial tightening force is reduced.

Then the second driving member having the second head with the outer diameter D4 is inserted from the proximal end of the hemostatic valve until the second head moves out of the outer sheath, and then the driving member is withdrawn until being outside the proximal end of the hemostatic valve. The inner diameter of the outer sheath D3 is further increased to D5 or the radial tightening force is further reduced.

In order to improve the effect of step-by-step pre-expansion, D0, D1 and D4 should meet an appropriate condition, for example, D1 is closer to D0 than D4.

For example, the difference between D4 and D0 is a reference value X, D1-D0=0.05X to 0.4X. For example, D1−D0=0.1X to 0. 2X. In one embodiment, D0=12F, D1=14F, and D4=26F.

According to the above-mentioned method for forming the interventional channel, the outer sheath is also pre-expanded. That is, a method for pre-expanding the outer sheath is also provided.

In one embodiment, the present disclosure further provides a transcatheter interventional system, which has opposing proximal and distal ends, including the guiding device of any of the above-mentioned embodiments and a delivery system. The delivery system includes a catheter assembly, a control handle connected to the proximal end of the catheter assembly and a prosthetic implant loaded in the distal end of the catheter assembly. The outer sheath successfully forms the interventional channel in the pre-expanded state. The operator operates the control handle to deliver the prosthetic implant into the human body through the catheter assembly through the interventional channel, or for simulation training in vitro.

After the guiding device of the present disclosure is delivered to a preset site in the human body, the driving member moves along the working direction to expand the outer sheath until the driving member is withdrawn from the human body to form the interventional channel. The operation of withdrawing the driving member from the human body and the expansion operation are completed at the same time to improve the efficiency. During the working process of the driving member, the resistance of the outer sheath to the driving member is reduced, so that the expansion operation is smoother. Moreover, compared with the existing pushing operation, the pulling operation is less difficult, and has lower requirements on the structural strength of the transmission part.

The technical features of the above embodiments can be arbitrarily combined, and not all possible combinations of the technical features of the above embodiments have been described for the sake of brevity of description. However, as long as there is no contradiction in the combination of these technical characteristics, such combination should be regarded as falling into the scope of this specification. When the technical features in different embodiments are shown in the same drawing, it can be considered that the drawing also discloses a combined embodiment of various embodiments involved.

The above-described embodiments only illustrate several embodiments of the present disclosure, and the description thereof is specific and detail, but should not be construed as limiting the scope of the patent disclosure. It should be noted that, for those of ordinary skill in the art, several modifications and improvements can be made without departing from the concept of the present disclosure, all of which fall into the protection scope of the present disclosure.

What is claimed is:

1. A guiding device for forming an interventional channel, having opposing proximal and distal ends and comprising:
an outer sheath being a radially deformable tube;
a hemostatic valve connected to the proximal end of the outer sheath; and
a driving member comprising a head and a transmission part, the driving member having an initial position and a working direction directed from the initial position toward the proximal end, wherein in the initial position, the head is outside the outer sheath and on a distal side of the outer sheath, and the transmission part is connected with the head and extends inside the outer sheath until being outside the proximal end of the outer sheath through the hemostatic valve, and wherein the head has a fixed shape, and when the driving member moves along the working direction, the head enters the outer sheath and drives a corresponding portion of the outer sheath to radially expand.

2. The guiding device of claim 1, wherein a tube wall of the outer sheath has a coiled structure, and has an expanded state in which the coiled structure is unfolded at a corresponding portion and a pre-expanded state in which the coiled structure is restored.

3. The guiding device of claim 1, wherein the outer sheath is made of elastic material.

4. The guiding device of claim 3, wherein a wall of the outer sheath comprises reinforced fibers.

5. The guiding device of claim 4, wherein the outer sheath comprises an inner film layer, a middle layer and an outer film layer arranged sequentially from inside to outside in a radial direction.

6. The guiding device of claim 5, wherein the middle layer is made of reinforced fibers, and made by braiding a plurality of filaments.

7. The guiding device of claim 1, wherein the outer sheath has relative to-be-expanded state and pre-expanded state, and the outer sheath in the pre-expanded state has a larger cross-sectional area and/or reduced radial tightening force than in the to-be-expanded state.

8. The guiding device of claim 7, wherein when the driving member is in the initial position, the outer sheath is in the to-be-expanded state; and when the driving member moves along the working direction, a portion of the outer sheath corresponding to the head and a portion of the outer sheath on a distal side of the head are in the pre-expanded state.

9. The guiding device of claim 8, wherein the outer sheath has a cross section in shape of a regular circle or ellipse, the head has a radial dimension D1, the outer sheath has an inner diameter D0 in the to-be-expanded state, and D1 is greater than D0.

10. The guiding device of claim 1, wherein the head comprises a first crown at its proximal end, and the first crown continuously expands from the proximal end toward a distal end.

11. The guiding device of claim 10, wherein in the initial position, a proximal end face of the first crown abuts against a distal opening of the outer sheath.

12. The guiding device of claim 10, wherein the first crown has a chamfered or tapered outer peripheral surface.

13. The guiding device of claim 10, wherein the head further comprises a second crown at its distal end which tapers from a largest diameter of the first crown toward the distal end.

14. The guiding device of claim 10, wherein the head further comprises a second crown at its distal end, and a main body between the first crown and the second crown which extends in an equal diameter.

15. The guiding device of claim 10, wherein the driving member further comprises a guide portion located at a distal end of the head, and the guide portion tapers to its own distal end.

16. The guiding device of claim 13, wherein the guiding device further comprises a guide portion located at a distal end of the head, and the guide portion tapers to its own distal end; and
the second crown has a greater inclination than the guide portion.

17. The guiding device of claim 1, wherein the head is generally spherical or ellipsoidal.

18. The guiding device of claim 1, wherein the head is provided with a lubricating layer on an outer periphery thereof.

19. The guiding device of claim 18, wherein in the initial position, the lubricating layer extends to a contact portion of the head with the outer sheath.

20. The guiding device of claim 1, wherein the driving member is formed by separate pieces and selected from one of the following arrangements:
a) the head and the transmission part are separate pieces in an axial direction;
b) the head comprises separate parts in a radial direction; and
c) the transmission part further comprises a separate handle at a proximal end of the hemostasis valve.
